(19) United States
(12) Patent Application Publication
(10) Pub. No.: US 2023/0364361 A1
(43) Pub. Date: Nov. 16, 2023

(54) APPARATUS FOR AGITATION OF MEDICAMENT FORMULATION

(71) Applicant: KINDEVA DRUG DELIVERY L.P., Woodbury, MN (US)

(72) Inventors: Andrew Henry, Bracknell (GB); Neale Griffiths, Bracknell (GB); Sabeel Ullah, Bracknell (GB); Katie Stoate, Bracknell (GB); Barzin Gavtash, Loughborough (GB); Philip Cocks, West Bridgford (GB); Paul Jolly, Bracknell (GB); Parfes Mohammed, Bracknell (GB); Lee Hodges, Clitheroe (GB)

(21) Appl. No.: 18/025,491

(22) PCT Filed: Sep. 9, 2021
(86) PCT No.: PCT/US2021/049587

§ 371 Date: Mar. 9, 2023

(30) Foreign Application Priority Data
Sep. 11, 2020 (GB) 2014292.3

(51) Int. Cl. A61M 15/00

(52) U.S. Cl. CPC A61M 15/002; A61M 15/0066; A61M 15/009; A61M 2205/50

(57) ABSTRACT

Various embodiments of a storage and dispensing apparatus for a pressurised metered dose inhaler and a method of utilizing such apparatus are disclosed. The apparatus includes a canister containing a mixture of a propellant and a therapeutic agent, an electronically actuated valve, and a controller configured to control the valve. The controller is further configured to actuate the valve to agitate the mixture within the canister, and actuate the valve to release a dose of the agitated mixture from the canister, where actuating the valve to agitate and actuating the valve to release a dose are discrete.

### CROSS-REFERENCE TO RELATED APPLICATION

This application claims the benefit of GB Patent Application No. 2014292.3, filed 11 Sep. 2020, which is incorporated herein by reference in its entirety.

### TECHNICAL FIELD

The present disclosure relates to an apparatus for agitation of a medicament formulation. More specifically, the present disclosure relates to an apparatus for agitation of a pressurised suspension or solution of a medicament in a propellant, in particular as used in inhalers.

### BACKGROUND ART

Inhalers are commonly used to treat a large array of medical conditions in both the human medical and veterinary fields. Inhalers come in many types, but prevalent among these are pressurised metered dose inhalers (pMDIs). These devices include a canister containing a mixture (typically a solution or a suspension) of a medicament and a propellant (typically a hydrofluoroalkane). The canister includes a metering valve, which is configured to receive a fixed volume from the canister into a metering chamber, and to expel the fixed volume when required. The metering valve typically includes a resiliently biased valve stem, which when depressed, expels the bolus of fluid from the valve chamber via a valve outlet orifice.

pMDIs include an actuator, typically constructed from a moulded plastics material, which serves to assist in actuation of the canister valve, and to deliver the medicament to the user. The actuator is a housing having a valve block that receives the canister valve, an expansion chamber, and a spray orifice or channel that delivers the expelled medicament to a nose- or mouthpiece that is inserted into the user's nose or mouth during use.

In use, the user first shakes the inhaler (including the canister) for several (e.g., 5-10) seconds to agitate the mixture and ensure that the medicament and propellant are evenly dispersed within the canister volume. This is particularly important in the case of suspensions, as the active ingredient can settle due to the density disparity between it and the propellant. The user then places the nose- or mouthpiece on or into the nose or mouth respectively, depresses the canister with respect to the actuator to actuate the valve, and inhales the expelled bolus of medicament to deliver the medicament to, e.g., the nasal cavity, lungs etc. In certain applications, for example paediatric or veterinary use, the inhaler may be used on the subject by a second party. Other devices, e.g., spacers may be used to aid inhalation of the medicament.

These devices can be used to treat a number of conditions—most commonly lung conditions such as asthma and COPD. Nasal inhalers are commonly used for, e.g., allergies. Their primary benefit is their ability to deliver the medicament directly to the tissue affected.

A problem with pMDIs, particularly those in which the medicament is in suspension in the propellant, is the tendency of the medicament and propellant to form an uneven or inhomogeneous mixture within the canister over time. The medicament can settle or float and depending on the orientation of the device in storage, can cause problems when the device is subsequently used. Hence users are instructed to shake the device before use.

Some users may forget to the shake the device, or not shake it for long enough (5-10 seconds is typically recommended). Also, if the device is used too long after shaking, the homogeneity of the contents may be partially lost. Also, some users may have conditions that prevent them from shaking the device in a comfortable fashion.

The pMDI industry is moving towards more ‘smart’ devices. These devices rely less on mechanical operation and instead are electrically powered and electronically controlled. Such devices meter on-demand, precise, tailored, doses of liquids. They incorporate sensors and electronics to optimise and monitor the dose delivery to the patient.

Problematically, such devices still tend to use the mechanical metering valve-type canisters. Therefore, although the device is ‘smart,’ it still requires the user to shake it before use.

It is an aim of the present disclosure to overcome, or at least mitigate, the above problem.

There have been several attempts in the prior art to overcome the need to shake the canister before use.

U.S. Pat. No. 7,185,648 discloses a medicament dispenser (e.g., a pMDI) having a container with an in-built agitator in the form of a moveable element such as a plunger or a wave energy generator. The agitator has a drive for driving the agitator independent any movement of the container.

U.S. Pat. No. 6,116,234 discloses a pMDI with a means for agitating the contents of the canister. Unlike US '648 above, the canister is conventional and rests on an agitating device having a polymeric spring and a piezoelectric crystal array. The crystal array is powered to provide a vibration to the canister shortly after depression that results in pressure waves in the contents. The mixing therefore occurs during dispensing of the container contents.

### SUMMARY OF INVENTION

According to a first aspect of the disclosure there is provided a storage and dispensing apparatus for a pressurised metered dose inhaler, the apparatus comprising:
a canister containing a mixture of a propellant and a therapeutic agent;
an electronically actuated valve;
a controller configured to control the valve;
wherein the controller is further configured to:
   actuate the valve to agitate the mixture within the canister; and
   actuate the valve to release a dose of the agitated mixture from the canister;
wherein actuating the valve to agitate and actuating the valve to release a dose are discrete.

Advantageously, utilising the valve to agitate the mixture removes the need for the user to remember to shake the canister before use. Further, using the valve avoids the need for a separate agitation device as seen in the aforementioned prior art. Still further, mixing can take place when the canister body and metering valve are in fluid communication. This is advantageous compared to US '234 because that device only mixes upon actuation, i.e., in the canister body and metering chamber as separate and distinct volumes.

The controller can be further configured to move the valve in a reciprocal motion to actuate the valve to agitate the mixture within the canister. By ‘reciprocal’ we mean moving in a first direction, then reversing that motion.

The controller can be further configured to move the valve in a further reciprocal motion to actuate the valve to release a dose of the agitated mixture from the canister after the valve has been moved in a reciprocal motion to actuate the valve to agitate the mixture within the canister.

The controller can be further configured to move the valve in an oscillatory motion to actuate the valve to agitate the mixture within the canister. By ‘oscillatory’ we mean a repeated reciprocal motion.

The valve can include a moveable valve member, and actuating the valve to agitate the mixture and actuating the valve to release a dose can be separated by a dwell period in which the valve member is stationary.

A flow restriction can be provided between the valve and the canister such that actuation of the valve to agitate the mixture causes a fluid jet to be created from the restriction into the canister to agitate at least a portion of the contents. The flow restriction can have an effective diameter between 0.7 and 4 mm.

In one or more embodiments:
the valve includes a moveable valve member;
where the valve member is moveable by a first distance to a condition in which fluid exits the canister to the valve when the valve is actuated to release a dose; and
where the valve member moves by a second distance less than the first distance such that no fluid exits the canister to the valve when the valve is actuated to agitate the mixture.

The valve can be a shuttle valve having a valve body and an axially moveable valve member.

The mixture within the canister can be agitated by motion of an axial end of the shuttle valve.

The valve can be a metering valve configured to release a predetermined volume of the mixture during actuating the valve to release a dose.

In one or more embodiments, the metering valve includes:
a metering chamber inlet in fluid communication with the canister;
a metering chamber outlet for release of a dose of mixture; and
a valve member whose position is controlled by the controller to move between:
   a rest position;
   an agitation position in which the metering chamber is sealed from the inlet;
   a filling position in which the metering chamber is in fluid communication with the inlet but sealed from the outlet; and
   an emptying position in which the metering chamber is in fluid communication with the outlet but sealed from the inlet.

In one or more embodiments:
the rest position and the emptying position are a first distance apart;
the rest position and the agitation position are a second distance apart, the second distance being greater than the first distance; and
the rest position and the filling position are a third distance apart, the third distance being greater than the second distance.

The apparatus can include an electrically powered actuator controlled by the controller.

The mixture can be a suspension of the therapeutic agent in the propellant.

The controller can be configured to only actuate the valve to release a dose within a predetermined time window after the valve has been actuated to agitate the mixture.

The valve can be resiliently biased to a closed condition by the pressure of the mixture within the container.

Various embodiments of the present disclosure can be particularly effective when the viscosity of the contents of the canister is between 1×10⁻⁴ and 5×10⁻⁴ Pa·s.

Various embodiments of the present disclosure can be particularly effective when the density ratio of the propellant to the therapeutic agent is between 0.4 and 1.3. Specifically, when the propellant/co-solvent is in the range 0.7 to 1.5 g/ml and the solid API/excipient has a true density in the range 1.2 to 1.6 g/ml.

According to a second aspect of the disclosure there is provided a method of actuating a storage and dispensing apparatus for a pressurised metered dose inhaler, the apparatus including:
a canister containing a mixture of a propellant and a therapeutic agent; and
a valve;
where the method includes:
   actuating the valve to agitate the mixture within the canister; and
   actuating the valve to release a dose of the agitated mixture from the canister;
   where actuating the valve to agitate and actuating the valve to release a dose are discrete.

The apparatus can include an electronic controller, and where actuating the valve to agitate and actuating the valve to release the dose are carried out by the controller.

The apparatus can be according to the first aspect.

According to a third aspect of the disclosure there is provided a method of delivery of a medicament to a human or animal including:
providing a pressurised metered dose inhaler having:
a storage and dispensing apparatus including:
   a canister containing a mixture of a propellant and a therapeutic agent; and
   a valve; and
a delivery portion for delivering the medicament to a human or animal;
   where the method includes:
actuating the valve to agitate the mixture within the canister; and
actuating the valve to release a dose of the agitated mixture from the delivery portion;
where actuating the valve to agitate and actuating the valve to release a dose are discrete.

### BRIEF DESCRIPTION OF DRAWINGS

Example devices according to the present disclosure will now be described with reference to the Figures in which:

FIG. 1 is a side, partially sectioned view of a first pMDI in accordance with the present disclosure;

FIG. 2 is a cross-section view of a metering valve of the pMDI of FIG. 1;

FIGS. 2a to 2g are detailed section views of a part of the pMDI of FIG. 1 with the valve in various conditions, where FIG. 2a shows the metering valve in a closed condition; FIG. 2b shows the metering valve in a valve closed/agitation condition; FIG. 2c shows the metering valve in a valve open/agitation condition; FIG. 2d shows the metering valve in a valve open condition; FIG. 2e shows the metering valve in a valve closed condition; FIG. 2f shows the metering valve in a valved closed condition; and FIG. 2g shows the metering valve in a valve closed condition with a metering chamber of the valve in fluid communication with an outlet orifice of the valve.

FIG. 3 is a flow chart of the method of operation of the pMDI of FIG. 1;

FIG. 4 is a displacement-time plot for a part of the pMDI of FIG. 1;

FIG. 5 is a flow chart of another embodiment of a method of operation of the pMDI of FIG. 1; and,

FIG. 6 is a displacement-time plot for a part of the pMDI of FIG. 1 of an alternative to FIG. 4 for a further variation of the first embodiment.

### DESCRIPTION OF EMBODIMENTS

### First Embodiment

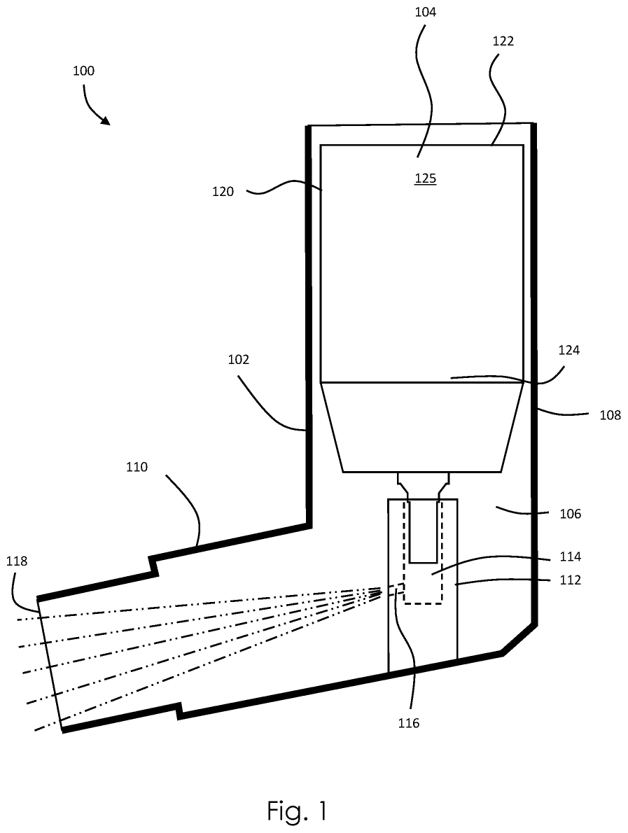

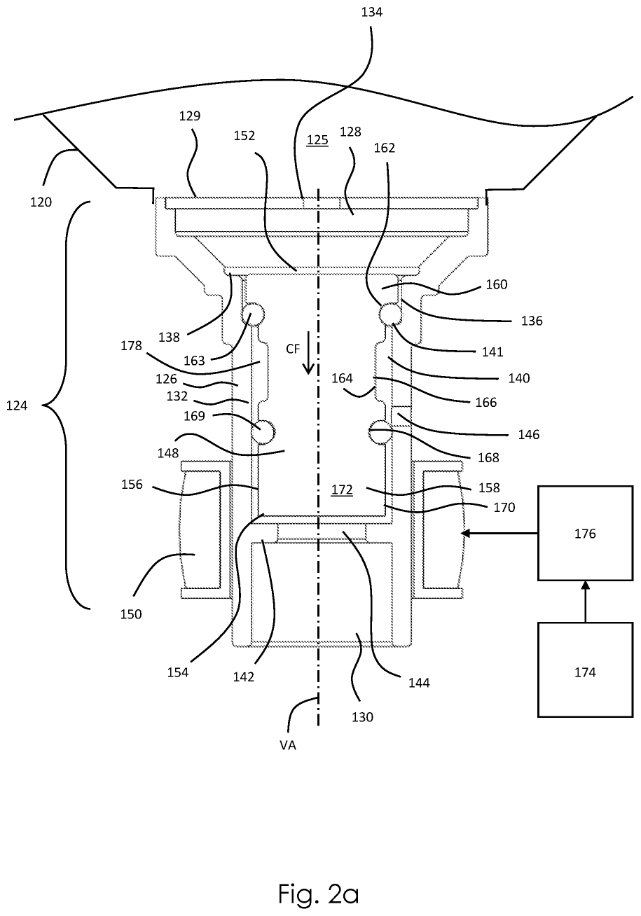

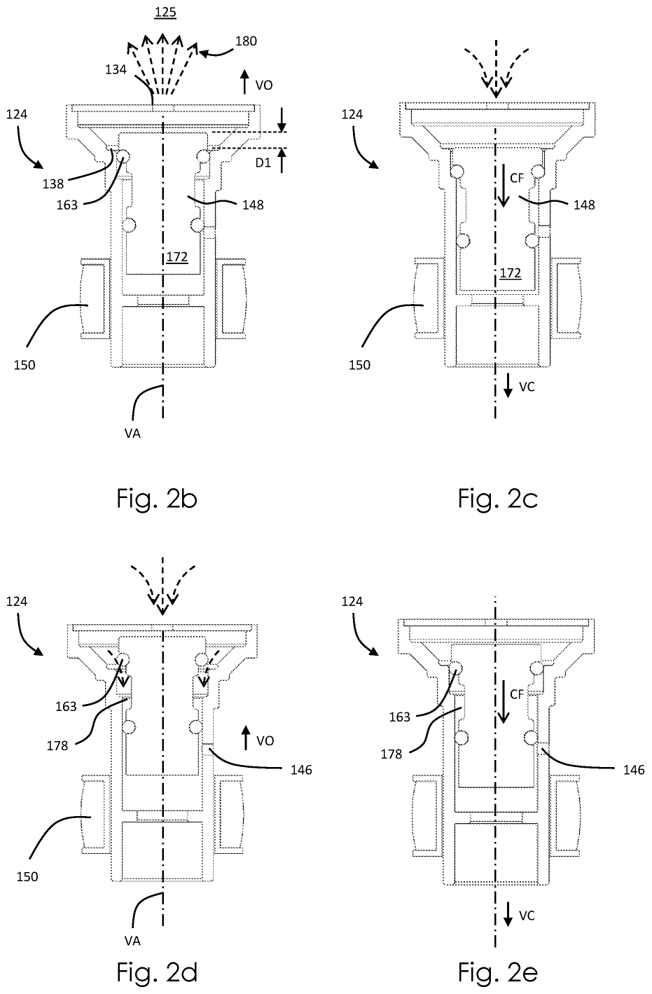

Referring to FIGS. 1 to 2g there is shown a pMDI 100 in accordance with the present disclosure.

### Configuration of the First Embodiment

The pMDI 100 includes a housing 102 and a canister 104. The pMDI 100 may be a ‘smart’ or electronically controlled device, although only the electronically controlled elements of the valve will be described here.

The housing 102 is a hollow structure defining an inner volume 106. The housing 102 is generally constructed from a moulded plastics material. The housing 102 defines a canister receiving portion 108 which is vertical in use, and a mouthpiece 110 extending from a lower end thereof in use, at an angle to the canister receiving portion. The mouthpiece 110 extends generally horizontally and downwardly in use relative to the canister receiving portion 108. The mouthpiece 110 has an outlet opening 118.

Where the canister receiving portion 108 and mouthpiece 110 meet, there is provided a canister mount 112 including an expansion chamber 114 and a spray orifice 116 in fluid communication therewith. The expansion chamber 114 is in fluid communication with an outlet from the canister valve (describe below) in use. The spray orifice 116 is directed towards the outlet opening 118 of the mouthpiece 110.

The canister 104 includes a sealed, cylindrical pressurised fluid container 120 having a base 122 (which appears at the top of FIG. 1 because the canister is inverted in use) and a metering valve 124 at the end opposite the base 122. The container 120 defines a pressurised fluid volume 125.

Turning to FIG. 2, the metering valve 124 is shown in more detail. The valve 124 is a shuttle valve and includes a valve housing 126, a valve member 148 and a solenoid electromagnet 150.

The valve housing 126 is generally cylindrical having a main valve axis VA. The valve housing 126 defines an inner surface 132 extending along axis VA defining a cylindrical fluid conduit. It has an inlet region 128 at a first end and a relief outlet 130 at a second end.

The inlet region 128 comprises an annular flange 129 defining a central inlet orifice 134. The inlet region 128 is adjacent a first valve-receiving portion 136 having a lower diameter, the region 134 and portion 136 being joined by a shoulder 138. A seal-bearing shoulder 141 is provided at a lower end of the first valve-receiving portion 136 leading to a narrower second valve receiving portion 140. The second valve receiving portion 140 terminates in an inner, radially extending annular flange 142 having an opening 144 therein and leading to the relief outlet 130.

A radially extending outlet orifice 146 is defined in the circumferential side wall of the valve housing 126.

The valve member 148 is a generally cylindrical, solid member including a first, circular, end face 152, a second, annular, end face 154 and a radially facing outer surface 156 therebetween. The second end face 154 has a magnet-receiving formation 158 defined therein.

The radially facing outer surface 156 defines a first portion 160 proximate the first face 152, adjacent to which is provided a first o-ring groove 162. A second portion 164 defines a circumferential, annular recess 166 which as described below, in use, defines part of a metering chamber. A second o-ring groove 168 is provided on the opposite side of the recess 166 to the first o-ring groove 162. A third portion 170 is provided adjacent the second o-ring groove 168 and the second face 154.

Two o-ring seals 163, 169 are positioned within the grooves 162, 168.

A permanent magnet 172 is provided within the magnet-receiving formation 158.

The solenoid electromagnet 150 encircles the valve housing 126 proximate the flange 142. An electrical power source 174 (in the form of a battery) and controller 176 are provided. The controller 176 is configured to control delivery of power from the source 174 to the electromagnet 150.

When assembled, the valve member 148 is moveable within the valve housing 126 such that it can move along the axis VA. An annular metering chamber 178 is defined between the valve member 148 and the inner surface 132.

### Operation of the First Embodiment

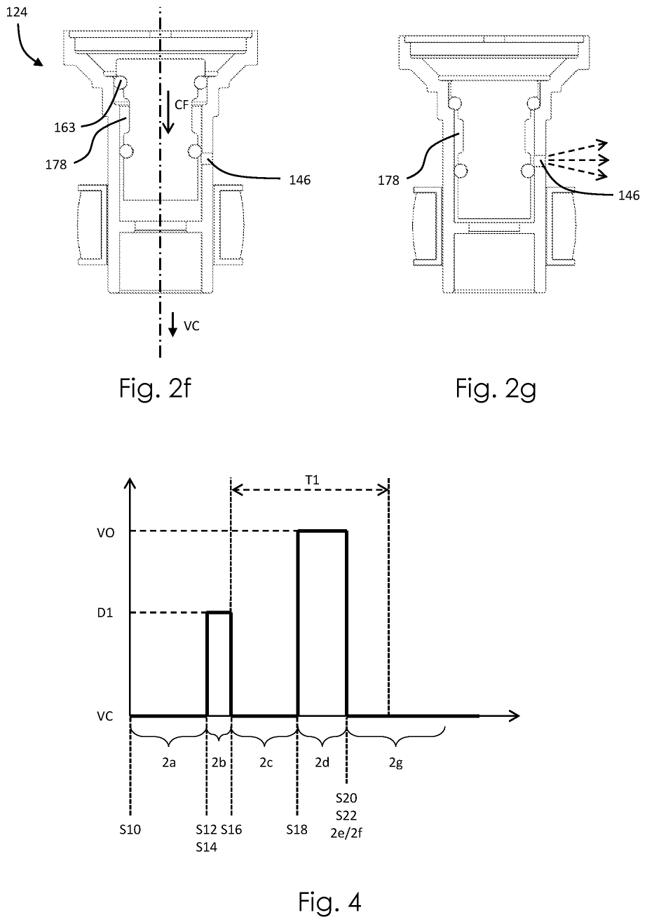

The operational steps of the first embodiment are shown in FIG. 3.

At step S10, the inhaler 100 is in the ‘valve closed’ condition. FIG. 2a shows the closed condition of the valve. In this condition, the electromagnet 150 is not energised, and the pressure of the fluid within the volume 125 creates a net downward closing force CF on the valve member 148 such that the o-ring 163 bears against the shoulder 141.

At step S12, when the user is ready to activate the inhaler 100, he or she provides a user input which sends a signal to the controller 174. The user input may be, e.g., a button or a touch screen. Upon receipt of the command, the controller 174 initiates the agitation step at S14.

The agitation step at S14 is designed to replace, or reduce the reliance on, the need for the user to shake the device 100 before use. During this step, the controller 174 energises the electromagnet 150. This energisation is configured to move the valve member 148 in a valve opening direction VO by a predetermined distance D1 to an ‘valve closed/agitation’ condition as shown in FIG. 2b. The distance D1 is sufficient to reduce the volume between the valve member 148 and the flange 129 and thus expel fluid from the orifice 134 in a jet 180 into the canister volume 125. The presence of this jet of fluid serves to induce mixing forces in the fluid in the canister. The distance D1 is however insufficient to get the o-ring seal 163 past the shoulder 138. Therefore, no fluid passage is provided between the volume 125 and the metering chamber 178.

The electromagnet 150 is de-charged and the valve member 148 returns to the closed condition in the valve closing direction VC under the closing force as shown in FIG. 2c and at step S16 as shown in FIG. 3. As this occurs, fluid is drawn back through the orifice 134.

At this stage the inhaler 100 is in an agitated state, with the contents of the volume 125 in a more homogenous/mixed condition.

At step S18 the controller 176 energises the solenoid electromagnet 150 to move the valve member 124 in the valve opening direction VO to a ‘valve open’ condition as shown in FIG. 2d, where the o-ring 163 has passed the shoulder 138 to place the metering chamber 178 in fluid communication with the volume 125. Because the chamber 178 is at atmospheric pressure, fluid is drawn into the chamber 178 from the higher-pressure canister volume 125. As the top o-ring 163 passes the shoulder 138 to open the metering chamber 178, the lower o-ring 169 passes the outlet orifice 146 to seal it from the metering chamber 178.

At step S20, the electromagnet 150 is de-charged and the closing force CF of the canister fluid pressure forces the valve member 148 in a valve closed direction VC as shown in FIGS. 2e to 2f. At the condition in FIG. 2e (the ‘metering chamber closed’ condition) the metering chamber 178 is sealed from both the canister volume 125 and the outlet orifice 146.

At step S22, the valve member 148 has moved back to the ‘valve closed’ condition in which the metering chamber 178 is in fluid communication with the outlet orifice 146 as shown in FIG. 2g. In this condition, the pressurised fluid in the chamber 178 can exit the outlet 146 and is delivered to the expansion chamber 114 and the spray orifice 116 (FIG. 1).

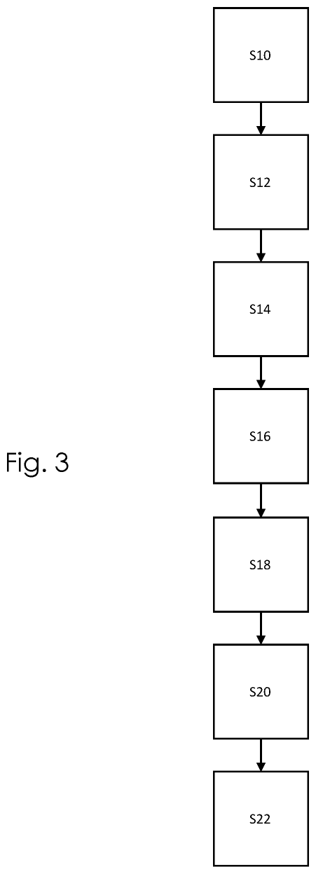

FIG. 4 shows valve displacement on the Y axis against time on the X axis. FIGS. 2a to 2f are referred to on the X-axis. It will be understood that valve movement is not instantaneous, as implied by the graph, but FIG. 4 provides a simplified representation of motion over time. The degree of travel required for agitation (D1) is less than the full travel to the VO condition. This is achieved by the controller 176 which provides a lower duration input signal to the electromagnet to ensure that the valve member 124 does not travel all the way to VO.

In this embodiment the agitation step is carried out followed by the dosage step automatically, i.e., the user is not required to provide an input to carry out dosage.

### Variations on the First Embodiment

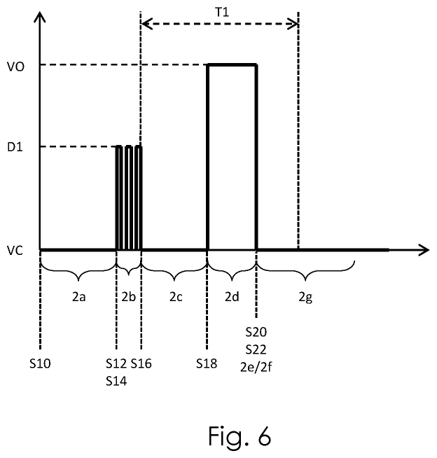

Turning to FIG. 6, an alternative to FIG. 4 is shown in which the agitation stage at 2b includes a plurality of pulses, rather than one single agitation pulse. This motion is oscillatory and may be akin to a square wave (as shown), or any other type of repeated reciprocal motion.

In an alternative embodiment, the controller 176 may provide a lower amplitude signal to the electromagnet during agitation, instead of, or in combination with, a lower duration signal. Either way, the signal is of lower overall energy.

In a still further embodiment, an interlock may be provided to inhibit travel of the valve member 124 beyond D1 until agitation has been carried out. This interlock may be mechanical (e.g., a member projecting into the valve to inhibit movement), or, e.g., electromagnetic (for example a further coil inhibiting movement of the permanent magnet).

In an alternative embodiment, the user is required to provide a further input for the dosage step at S18. The controller 176 includes an interlock function that prevents step S18 from being carried out before the agitation step at S14. It may be the case that for devices with touchscreens that the option of S18 is simply not available until agitation has been performed. In other embodiments, the ‘dosage’ button may be deactivated until agitation has been carried out.

It will be noted that the effect of the agitation decreases over time T1, because the contents of the canister will tend to naturally de-homogenise under gravity. Therefore, in one or more embodiments, the step S18 can be carried out before T1 expires (see FIG. 4).

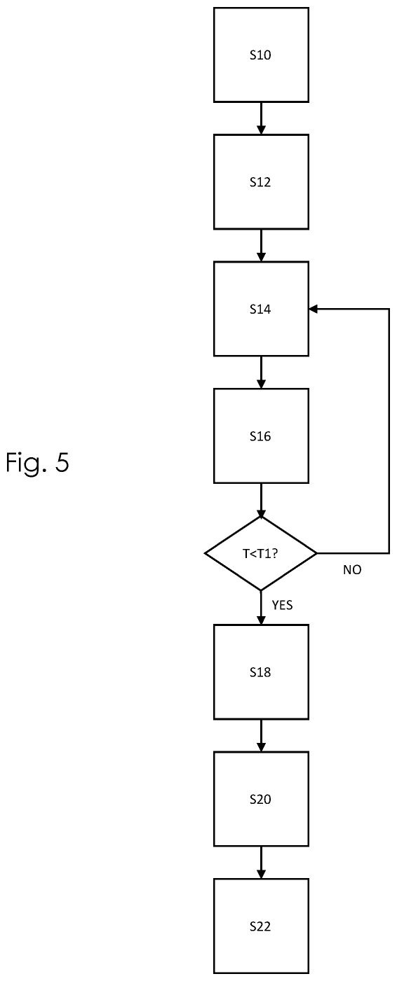

The controller 176 may also include a timer such that the step S18 must be carried out within T1 (predetermined during set up), else a further agitation step S18 may be required. This is shown in FIG. 5.

Instead of a mouthpiece 110, the pMDI 100 may be provided with a nosepiece for insertion into a nostril. It may also be provided with other types of outlets, for example a spacer or a formation for attachment of other inhalation related devices.

The embodiment mentions a single movement of the valve member 148 to cause agitation. It is noted that multiple movements, for example in a reciprocating or vibrating manner may be provided for agitation.

The first embodiment features a solenoid/electromagnetically actuated valve member. It will be noted that the actuation mechanism can take other forms within the scope of the present disclosure. For example, the shuttle valve may be mechanically driven via a gear train from a rotational drive (such as a motor). It may also be actuated by a piezoelectric actuator, or even a hydraulic or pneumatic actuator.

The first embodiment has focused on implementation using a linear shuttle valve, but this principle can be applied to any valve (specifically considering rotational valves too) where there is the ability to provide valve movement before metering/firing.

It is envisaged that added geometrical features may be provided to stir the fluid. For example, on a rotational-type valve, fins or paddles may be provided adjacent to the canister volume to agitate the contents.

In an alternative embodiment, an expansion chamber (114 in the first embodiment) is not provided.

In the first embodiment, the fluid is expelled from the orifice 134 in a jet 180 into the canister volume 125. The presence of this jet of fluid serves to induce mixing forces in the fluid in the canister. In an alternative embodiment, the annular flange 129 defining a central inlet orifice 134 may be omitted, such that the end face 152 of the valve member 148 is in direct contact with the main volume of the canister. In this embodiment, the end face 152 of the valve member serves to impart a pressure wave to the canister contents to enable mixing to take place.

The present disclosure may be particularly effective for suspensions, i.e., canister contents in which particles are suspended in a fluid medium. Such mixtures tend to separate, i.e., the suspended contents tend to sink or float depending on the relative densities of the substances. It will also be noted that the present disclosure may be utilised for canisters where the contents are in solution and is effective in re-homogenising solutions with partial immiscibility in particular.

1. A storage and dispensing apparatus for a pressurised metered dose inhaler, the apparatus comprising:
a canister containing a mixture of a propellant and a therapeutic agent;
an electronically actuated valve;
a controller configured to control the valve;
wherein the controller is further configured to:
actuate the valve to agitate the mixture within the canister; and
actuate the valve to release a dose of the agitated mixture from the canister;
wherein actuating the valve to agitate and actuating the valve to release a dose are discrete.

2. An apparatus according to claim 1, wherein the controller is further configured to move the valve in a reciprocal motion to actuate the valve to agitate the mixture within the canister.

3. An apparatus according to claim 2, wherein the controller is further configured to move the valve in a further reciprocal motion to actuate the valve to release a dose of the agitated mixture from the canister after the valve has been moved in a reciprocal motion to actuate the valve to agitate the mixture within the canister.

4. An apparatus according to claim 2, wherein the controller is further configured to move the valve in an oscillatory motion to actuate the valve to agitate the mixture within the canister.

5. An apparatus according to claim 1, wherein the valve comprises a moveable valve member, and wherein actuating the valve to agitate the mixture and actuating the valve to release a dose are separated by a dwell period in which the valve member is stationary.

6. An apparatus according to claim 1, wherein a flow restriction is provided between the valve and the canister, such that actuation of the valve to agitate the mixture causes a fluid jet to be created from the restriction into the canister to agitate the contents.

7. (canceled)

8. An apparatus according to claim 1, wherein:
the valve comprises a moveable valve member;
wherein the valve member is moveable by a first distance to a condition in which fluid exits the canister to the valve when the valve is actuated to release a dose; and
wherein the valve member moves by a second distance less than the first distance such that no fluid exits the canister to the valve when the valve is actuated to agitate the mixture.

9. An apparatus according to claim 1, wherein the valve is a shuttle valve comprising a valve body and an axially moveable valve member.

10. (canceled)

11. An apparatus according to claim 1, wherein the valve is a metering valve configured to release a predetermined volume of the mixture during actuating the valve to release a dose.

12. An apparatus according to claim 11, wherein the metering valve comprises:
a metering chamber inlet in fluid communication with the canister;
a metering chamber outlet for release of a dose of mixture; and
a valve member whose position is controlled by the controller to move between:
a rest position;
an agitation position in which the metering chamber is sealed from the inlet;
a filling position in which the metering chamber is in fluid communication with the inlet but sealed from the outlet; and
an emptying position in which the metering chamber is in fluid communication with the outlet but sealed from the inlet.

13. An apparatus according to claim 12, wherein:
the rest position and the emptying position are a first distance apart;
the rest position and the agitation position are a second distance apart, the second distance being greater than the first distance; and
the rest position and the filling position are a third distance apart, the third distance being greater than the second distance.

14. (canceled)

15. An apparatus according to claim 1, wherein the mixture is a suspension of the therapeutic agent in the propellant.

16. An apparatus according to claim 1, wherein the controller is configured to only actuate the valve to release a dose within a predetermined time window after the valve has been actuated to agitate the mixture.

17. An apparatus according to claim 1, wherein the valve is resiliently biased to a closed condition by the pressure of the mixture within the container.

18. An apparatus according to claim 1, wherein the viscosity of the contents of the canister is between 1×10⁻¹ and 5×10⁻⁴ Pa·s.

19. An apparatus according to claim 1, wherein the density ratio of the propellant to the therapeutic agent is between 0.4 and 1.3

20. A method of actuating a storage and dispensing apparatus for a pressurised metered dose inhaler, the apparatus comprising:
a canister containing a mixture of a propellant and a therapeutic agent; and
a valve;
wherein the method comprises:
actuating the valve to agitate the mixture within the canister; and
actuating the valve to release a dose of the agitated mixture from the canister;
wherein actuating the valve to agitate and actuating the valve to release a dose are discrete.

21. The method of actuating according to claim 20, wherein the apparatus comprises an electronic controller, and wherein actuating the valve to agitate and actuating the valve to release the dose are carried out by the controller.

22. The method of actuating according to claim 20, wherein the apparatus is according to claim 1.

23. A method of delivery of a medicament to a human or animal comprising:
providing a pressurised metered dose inhaler having:
a storage and dispensing apparatus comprising:
a canister containing a mixture of a propellant and a therapeutic agent; and
a valve; and
a delivery portion for delivering the medicament to a human or animal;
wherein the method comprises:
actuating the valve to agitate the mixture within the canister; and
actuating the valve to release a dose of the agitated mixture from the delivery portion;
wherein actuating the valve to agitate and actuating the valve to release a dose are discrete.
